(19) United States
(12) Patent Application Publication
(10) Pub. No.: US 2025/0339194 A1
(43) Pub. Date: Nov. 6, 2025

(54) CRYO NOZZLE

(71) Applicant: AtriCure, Inc., Mason, OH (US)

(72) Inventors: Robert KLEIN, Mason, OH (US); Christopher William WIDENHOUSE, Mason, OH (US); Tatiana SEDLAK, Mason, OH (US)

(73) Assignee: AtriCure, Inc., Mason, OH (US)

(21) Appl. No.: 19/266,851

(22) Filed: Jul. 11, 2025

Related U.S. Application Data
(63) Continuation of application No. PCT/US2025/012026, filed on Jan. 17, 2025.
(60) Provisional application No. 63/621,849, filed on Jan. 17, 2024.

(51) Int. Cl. A61B 18/02; A61B 18/00

(52) U.S. Cl. CPC A61B 18/02; A61B 2018/00023; A61B 2018/00101; A61B 2018/00577; A61B 2018/0293

(57) ABSTRACT

Disclosed herein are systems and devices comprising a cryogenic device for ablating tissue. The cryogenic device can comprise a handle and an elongated shaft comprising a distal end and a proximal end. The elongated shaft can extend from the handle at the proximal end. The cryogenic device can further comprise an end effector at the distal end of the elongated shaft, wherein the end effector comprises a percutaneous probe. The cryogenic device can further comprise an inlet line tube within the elongated shaft. The cryogenic device can further comprise a nozzle at a distal end of the inlet line tube, wherein the nozzle comprises a plurality of orifices to deliver fluid through the inlet line tube to the end effector.

### CROSS-REFERENCE TO RELATED APPLICATIONS

This application is a continuation of PCT Application No. PCT/US2025/012026, filed Jan. 17, 2025, which claims benefit of U.S. Provisional Application No. 63/621,849 filed Jan. 17, 2024, the entirety of which are incorporated by reference.

### FIELD OF TECHNOLOGY

The present disclosure relates generally to the field of cryogenic devices, and more specifically, cryogenic devices with various nozzle configurations.

### BACKGROUND

It is known to treat cardiac arrhythmias by creating one or more lines of scar tissue or ablation in the heart tissue to block errant electrical signals. The present application is directed to a cryogenic surgical instrument and, more particularly, to a cryogenic probe or cryoprobe, for creating lines of ablation on cardiac tissue for the treatment of cardiac arrythmia, such as atrial fibrillation. It is also known to perform nerve block, or localized analgesia, by freezing nerves and the tissue surrounding nerves to between −10° C. and −90° C.

Current cryogenic devices function by applying high pressure differential across a small nozzle that is located at the end of a metal probe. The N₂O, CO₂, or other gas expands as it goes through a small nozzle into the boiler chamber. During expansion, primarily due to the Joule-Thomson Effect, there is significant irreversible pressure work done. Secondary cooling comes from reversible ideal gas expansion and vapor-liquid condensation or vapor-solid freezing. The lower pressure gas then flows into the interior of the metal probe and back through the exhaust port.

Current devices often have blunt tubes that are pressed against cardiac or internal tissue. Other devices can have ball tips that are pressed against internal tissue. Other devices can have a two-sided prong that is used to grab and press against dissected nerve bundles. However, some devices do not provide for power and efficiency that results in efficient expansion of the vapor and more focused jetting of the vapor onto the end effector.

Therefore, there remains a need for methods and devices for cryogenic devices that protect surrounding tissue while cooling target tissue efficiently and consistently.

### SUMMARY

Disclosed herein are systems and devices comprising a cryogenic device for ablating tissue. The cryogenic device can comprise a handle and an elongated shaft comprising a distal end and a proximal end. The elongated shaft can extend from the handle at the proximal end. The cryogenic device can further comprise an end effector at the distal end of the elongated shaft, wherein the end effector comprises a percutaneous probe. The cryogenic device can further comprise an inlet line tube within the elongated shaft. The cryogenic device can further comprise a nozzle at a distal end of the inlet line tube, wherein the nozzle comprises a plurality of orifices to deliver fluid through the inlet line tube to the end effector.

The nozzle can comprise a converging portion and a diverging portion. The converging portion can be proximal to the diverging portion. The cryogenic device can further comprise a plate within the inlet line tube. The plate can extend radially and can comprise the plurality of orifices such that the plurality of orifices faces in a longitudinal direction. The percutaneous probe can comprise a closed needle. The cryogenic device can further comprise an inner shaft concentrically between the elongated shaft and the inlet line tube. The inner shaft and the inlet line tube can comprise an exhaust path therebetween. The end effector can be configured to cool tissue when cryogenic fluid is introduced to the end effector. The plurality of orifices can each be staggered along a length of the end effector.

In some variations, the cryogenic device can comprise an inner shaft concentrically between the elongated shaft and the inlet line tube. A vacuum insulated space can be between the inner shaft and the elongated shaft.

In some variations, the vacuum insulation layer can be configured to create an insulated portion of the needle such that the insulated portion does not damage tissue in contact with the needle. The end effector can be configured to ablate the tissue at a treatment portion, wherein the treatment portion is separate from the insulated portion. In some variations, a method for ablating tissue is disclosed, the method comprising advancing an elongated shaft percutaneously to a target location, the elongated shaft comprising an inner shaft concentrically within the elongated shaft, the inner shaft comprising an end effector at the distal end of the inner shaft having a needle, wherein a vacuum insulation layer is between the inner shaft and the elongated shaft and is configured to create an insulated portion of the needle such that the insulated portion does not damage tissue in contact with the needle; positioning the end effector such that a treatment portion of the end effector separate from the insulated portion is configured to ablate tissue at the target location; introducing a fluid through the elongated shaft and into the end effector; and ablating the target location with the end effector at the treatment portion.

### BRIEF DESCRIPTION OF THE DRAWINGS

FIG. 1A illustrates a cryogenic device in accordance with one variation of the invention.

FIG. 1B illustrates an elongated shaft in accordance with the cryogenic device of FIG. 1A.

FIG. 2A illustrates a cross-sectional view of an elongated shaft with a converging nozzle according to one variation of the invention.

FIG. 2B illustrates a cross-sectional view of an elongated shaft with a converging-diverging nozzle according to one variation of the invention.

FIGS. 3A to 3C illustrate cross-sectional views of various elongated shafts with multi-orifice nozzles.

FIG. 4A illustrates another variation of the elongated shaft having multiple nozzles at a distal end of the elongated shaft.

FIG. 4B illustrates a cross-sectional view of the elongated shaft having nozzles extending within the end effector at a distal end.

FIG. 5 illustrates another variation of the elongated shaft having multiple nozzles on an end plate of the elongated shaft.

FIG. 6 illustrates a system for using the cryogenic device.

### DETAILED DESCRIPTION

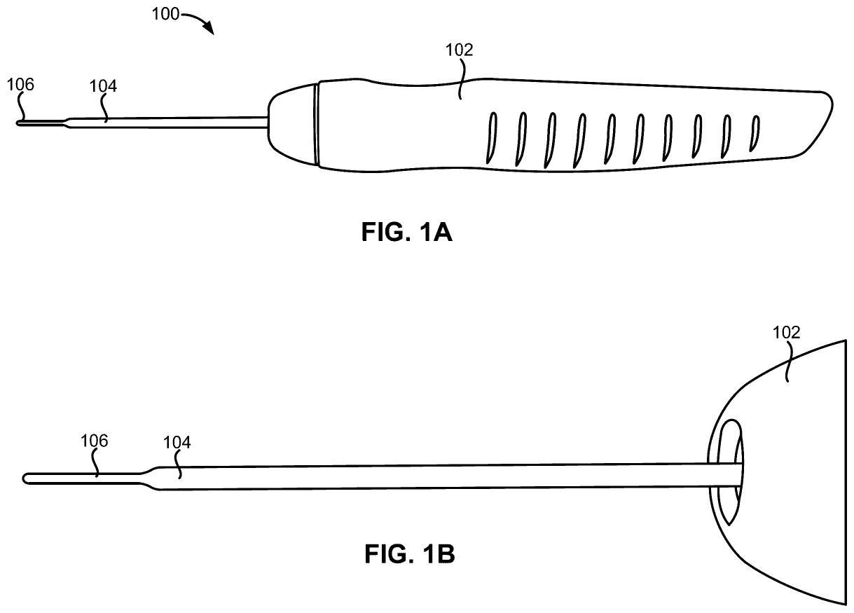

FIG. 1A illustrates a cryogenic device 100 in accordance with one variation of the invention. The cryogenic device 100 can comprise a handle 102 an elongated shaft 104, and an end effector 106 at a distal end of the elongated shaft 104. The elongated shaft 104 can comprise a proximal end coupled to the handle 102 such that the elongated shaft 104 extends from the handle 102.

The cryogenic device 100 can be used to ablate the endocardium or epicardium of the heart. The freezing of the cardiac tissue can cause an inflammatory response (cryonecrosis) that blocks the conduction of electrical pulses.

In some variations, the cryogenic device 100 can be used in a nerve block procedure, such as for pain management for the intercostal regions (i.e., between the ribs), thoracic nerves post-mastectomy, nerves associated with pain during or after lower and upper limb amputation, nerves associated with pain during or after joint replacements, nerves associated with migraine pain, nerves associated with biopsy pain, chronic nerve pain throughout the body, and similar applications. This can include nerves within the body cavity (such as intercostal or thoracic nerves), nerves accessible during amputation, and nerves accessible from the exterior of the body such as within or under the skin or eyes.

In some variations, the cryogenic device 100 can be used in a nerve block procedure targeting nerves post-amputation.

In some variations, the cryogenic device 100 can comprise a tissue-contacting probe for performing localized analgesia.

All materials used in the cryogenic device 100 that are exposed to the cryofluid may be compatible with the cryofluid used in the device, and components intended for patient contact may be biocompatible. The cryogenic device (and its packaging) can also be radiation stable and ethylene oxide stable via gamma sterilization, x-ray sterilization, e-beam sterilization, and ethylene oxide methods, among other methods.

As seen in the close-up view of FIG. 1B, the end effector 106 can be positioned at the distal end of the elongated shaft 104 and can be configured to cool tissue (for example, for cryoablation or cryoanalgesia) when cryogenic fluid is introduced to the end effector 106. The end effector 106 can generate surface temperatures below −20° C. When the end effector 106 is applied to the tissue to be treated, freezing of tissue coming into direct contact with the end effector 106 results. Surrounding tissue is sequentially frozen by the withdrawal of heat from the tissue as the end effector 106 maintains contact with tissue over time.

The end effector 106 can be constructed of any number of aluminum alloys or stainless steel alloys, which can withstand high pressure, have sufficient thermal conductivity, and also good biocompatibility. Alternatively, any material that can withstand the internal pressures and temperatures of the cryo process may be used, including metals, plastics, elastomers, and ceramics. These materials can also be coated with a thin layer of low-friction coating to reduce sticking while not significantly impacting the efficiency of the end effector 106.

The end effector 106 can have a smooth exterior surface for contacting the tissue to be ablated. The distal tip of the end effector 106 can be closed and form a blunt, atraumatic, generally hemispherical shape. This may be accomplished by limiting the opening in the end effector 106, by casting, spin forming, etc.

Surfaces of the cryogenic device 100 that are not intended for patient contact may be insulated for the protection of both non-target tissue and the user. To this end, the interior elongated shaft 104 can create an insulative pocket or gap that serves to insulate portions of the end effector 106, thus protecting adjacent non-treated tissue from freezing tissue that may come into contact with the exposed portion of the sleeve. Similarly, the handle 102 can provide an insulated surface to hold the probe tube in position while manipulating the end effector.

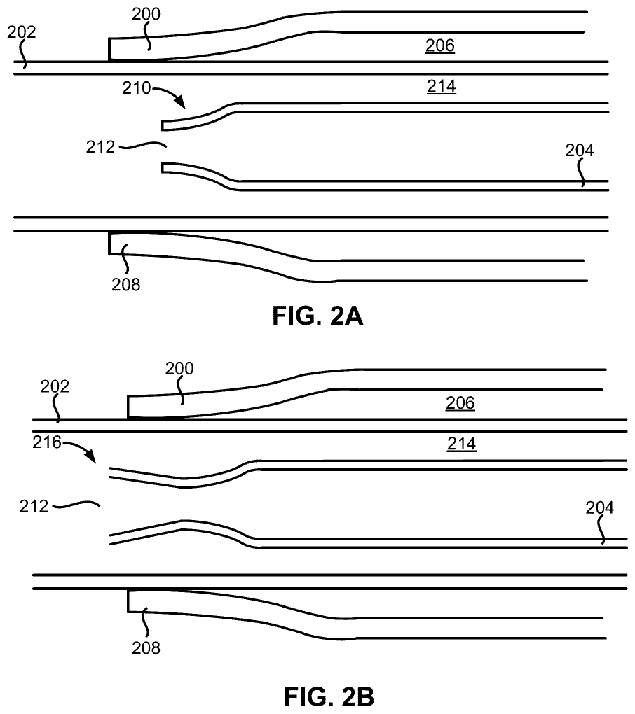

FIG. 2A illustrates a cross-sectional view of an elongated shaft 104 with a converging nozzle according to one variation of the invention. In this variation, the elongated shaft comprises an outer shaft tube 200, an inner shaft tube 202 concentrically within the outer shaft tube 200, and an inlet line tube 204 concentrically within the inner shaft tube 202. The inner shaft tube 202 can transition to the end effector 106 or the end effector 106 can be placed on a distal end of the inner shaft tube 202.

The outer shaft tube 200 and the inner shaft tube 202 can create an insulative layer 206 therebetween for a vacuum to be applied within, providing thermal insulation to the elongated shaft 104 by eliminating air within the space. Since the end effector 106 can be placed percutaneously into tissue to target the freezing of specific tissue in a specific location, insulating the elongated shaft 104 in all locations except where the desired freeze takes place is important to avoid damaging non-targeted tissues. The insulative layer 206 can also localize a freeze zone of the end effector 106 to a specific location.

In some variations, the insulative layer 206 can comprise infrared-reflective material to minimize heat transfer.

In some variations, the insulative layer 206 can be composed of an alternate gas, liquid, or solid that provides thermal insulation.

Surfaces of the cryogenic device 100 that are not intended for patient contact can be insulated for the protection of both non-target tissue and the user. The insulative layer 206 can thus create an air pocket that serves to insulate the portion of the probe tube proximal to the end effector 106, thus protecting adjacent non-treated tissue from freezing tissue that may come into contact with the exposed portion of the elongated shaft 104. Similarly, the handle 102 can provide an insulated surface to hold the probe tube in position while manipulating the end effector 106.

The insulative layer 206 can create a localized treatment portion or freeze portion at a distal end of the end effector 106 that is advantageous in combination with a percutaneous probe in that it allows the probe tip to be placed through tissue and freeze only the distal-most end while avoiding cryogenic damage to remaining tissue in contact with the end effector 106 at the insulated region(s). To this end, the outer shaft tube 200 and parts of the end effector 106 can be vacuum insulated while maintaining its structural integrity and flexibility for advancing through the anatomy.

In manufacturing, the vacuum can crimp and braze the outer shaft tube 200 to create a reduced diameter region 208 where the elongated shaft 104 transitions to the end effector 106 in part to improve the insulation of the end effector 106.

The inlet line tube 204 can comprise an opening or orifice 212 at a distal end through which fluid can pass through the inlet line tube 204 to the end effector 106 at a nozzle 210. The orifice 212 can be positioned at a reduced diameter region 208 to create a venturi effect as the fluid flows through the inlet line tube 204. The inlet line tube 204 and nozzle 210 can be circular, rectangular, star shaped, or any other shape.

The nozzle 210 can comprise a round tube that collapses to a smaller inner diameter. Fluid can flow through the inlet line tube 204 and can accelerate through the converging portion of the nozzle 210 where the flow rate is the highest. Fluid then expands after exiting the nozzle 210 into the end effector 106. To create the pressure differential, the flow rate of fluid reaches the speed of sound (Mach 1) in the smallest inner diameter of the nozzle 210 and for a short distance as it enters the end effector 106.

A Joule-Thomson effect can be formed inside the end effector 106 where the cryofluid undergoes expansion. The Joule-Thomson effect is created by the expansion of gas that occurs as the cryofluid moves through the small orifice from each of the high-pressure supply tubes into the low-pressure expansion chamber comprised by the elongated shaft 104. In some variations, the pressure differential at the nozzle for the use of N₂O and CO₂ is typically between about 100 psi and about 1000 psi. Temperatures within the elongated shaft 104 can be sufficiently cold so that surface temperatures of the end effector 106 may reach less than −20° C. in order to achieve cryogenic nerve block when nitrous oxide or carbon dioxide gas is used as the cryofluid.

The inlet line tube 204 and the inner shaft tube 202 can create an exhaust space 214 therebetween to exhaust the elongated shaft 104 during use. The exhaust space 214 can provide a path for the elongated shaft 104 to be relieved of unwanted fluid or debris during or after use.

FIG. 2B illustrates a cross-sectional view of an elongated shaft with a converging-diverging nozzle according to one variation of the invention. As seen in FIG. 2B, the orifice 212 can be positioned at a nozzle 216 that comprises a converging portion that collapses to a smaller inner dimension, and then opens distally into a cone-like nozzle at a diverging portion. Fluid can flow through the inlet line tube 204 and can accelerate through the converging portion of the nozzle 216 and then expand in a controlled and focused manner through the diverging portion of the nozzle 216 to the end effector 106. The converging-diverging nozzle 216 can provide a high velocity and thus a more efficient expansion of the fluid as well as a more focused jetting of the vapor onto the end effector 106.

The pressure differential, typically exceeding 500 psi but dependent on the size of the nozzle, can cause the flow rate of fluid to reach the speed of sound (Mach 1) in the smallest inner diameter of the nozzle 216. The fluid then continues to increase above Mach 1 to supersonic flow rates in the diverging portion of the nozzle 216. The flow rate decreases after the fluid exits the diverging nozzle as fluid moves into the end effector 106.

In some variations, the pressure differential can be lower resulting in a flow rate below Mach 1, but the Joule-Thompson effect can still be effective at cooling the fluid and the surrounding area. In this case, the converging-diverging nozzle still leads to a more efficient expansion of the fluid as well as a more focused jetting of the vapor onto the end effector 106 relative to a straight nozzle.

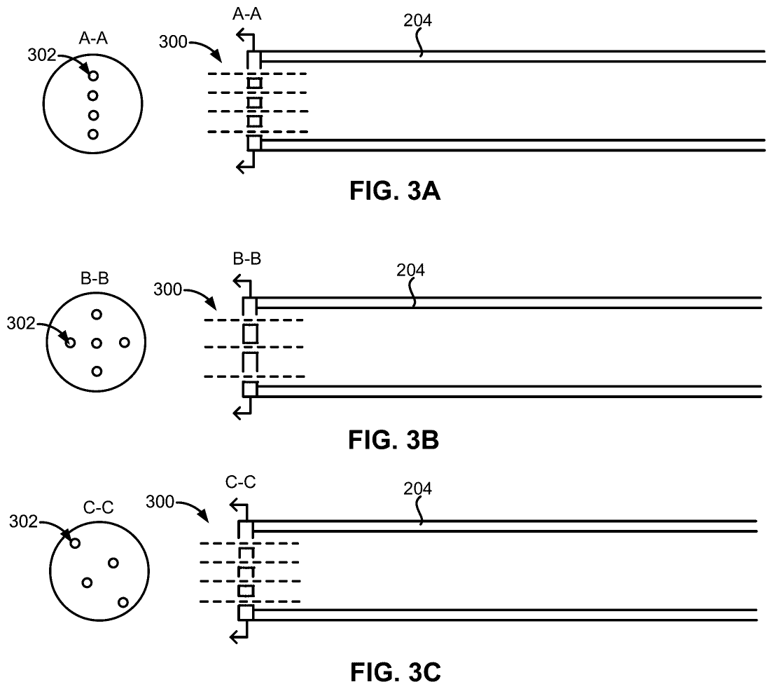

FIGS. 3A to 3C illustrate cross-sectional views of various elongated shafts with multi-orifice nozzles. In these variations, the nozzle 300 can comprise multiple orifices 302 built into the distal end of the inlet line tube 204 in various configurations. FIG. 3A illustrates four nozzles positioned radially across a cross-section A-A of the inlet line tube 204. FIG. 3B illustrates a variation of a nozzle 300 having five orifices 302 at the distal end of the inlet line tube 204 positioned in a cross-shape as seen in cross-section B-B. FIG. 3C illustrates a variation of a nozzle 300 having four orifices 302 at the distal end of the inlet line tube 204 positioned in a sporadic configuration as seen in cross-section C-C. It should be understood that any number of orifices can be used in any of the configurations shown.

Each of these orifices can function as a nozzle, with the pressure differential leading to a high flow rate of gas or liquid such as N₂O or CO₂ through the orifices, and thereby generating a cooling effect by way of the Joule-Thompson effect. The pressure differential can be sufficient to cause the flow rate to reach Mach 1 within the nozzle, or some lower pressure differential may still effectively induce the Joule-Thompson effect.

The inlet line tube 204 and nozzle can be circular, rectangular, star shaped, or any other shape. The inlet line tube 204 can comprise an outer diameter of about 0.016 inches to about 0.021 inches and a wall thickness of about 0.002 inches. The inner shaft tube 202 can comprise an outer diameter of about 0.032 inches to about 0.042 inches and a wall thickness of about 0.003 inches to about 0.004 inches. The outer shaft tube 200 can comprise an outer diameter of about 0.065 inches and a wall thickness of about 0.005 inches. The orifices of the nozzle can comprise an inner diameter of about 0.008 inches to about 0.0095 inches (e.g., 0.009 inches).

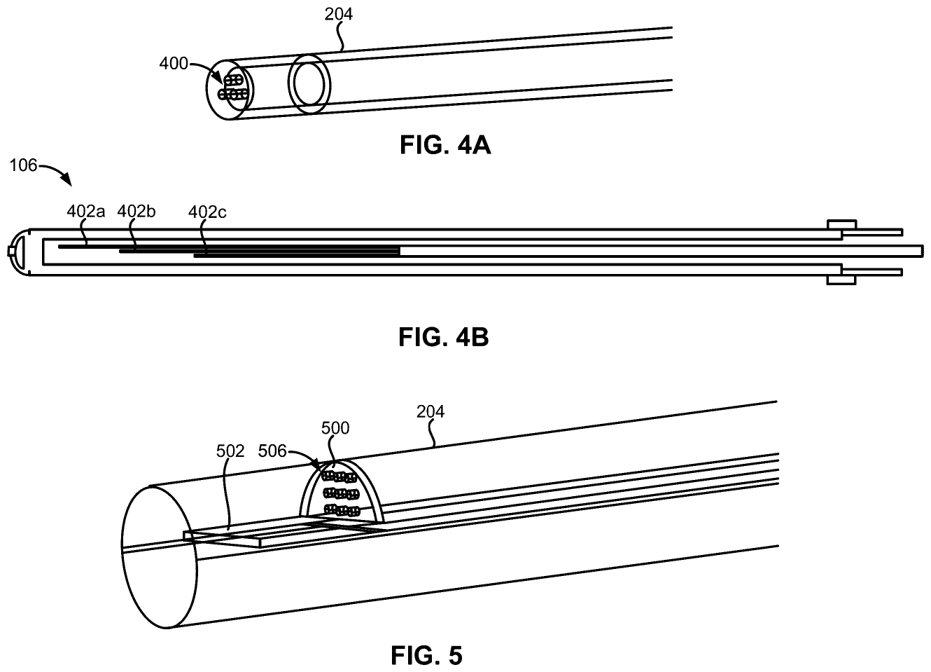

FIG. 4A illustrates another variation of the elongated shaft having multiple nozzles at a distal end of the elongated shaft. In this variation, three orifices 400 can be positioned at the distal end of the inlet line tube 204. The three orifices 400 can be configured in a triangular shape along an axial cross-section of the inlet line tube 204.

By adding multiple nozzles at the same location, it is possible to increase the total cooling power per nozzle unit area. Total cooling power scales linearly with the number of nozzles, and accordingly, an increase in nozzles increases the efficiency per nozzle unit area. In this variation, three 0.229 mm nozzles can provide cooling power per unit area that is about twice that of a larger single nozzle with the same total surface area. This variation increases the total flow rate at the end effector 106 as well as cools the end effector 106 at a fast rate such that lower pressure can be used to achieve adequate cooling power.

FIG. 4B illustrates a cross-sectional view of the elongated shaft having nozzles 402a, b, c extending within the end effector 106 at a distal end. The end effector 106 in this variation can comprise three separate nozzles 402a, b, c that extend from orifices of the inlet line tube 204 in a staggered configuration. The distal end of each nozzle 402a, b, c is staggered by about 10 mm to spread cooling along the length of the end effector 106.

FIG. 5 illustrates another variation of the elongated shaft having multiple nozzles on an end plate 500 within the inlet line tube 204. The end plate 500 can be shaped as a semicircle to conform with an inner surface of the inlet line tube 204 and can extend perpendicular from an axial plate 502 which extends flat through the inlet line tube 204. A total of nine nozzles 506 can be placed on the end plate 500 facing along the longitudinal axis of the elongated shaft 104. The nine nozzles 506 can be configured in an array on the end plate 500 as seen in FIG. 5. In other variations, twenty-five nozzles can be placed on the end plate 500.

In some variations, the end effector 106 can comprise a percutaneous needle or probe, with the purpose of either freezing a smaller region, or puncturing a tissue surface (skin, internal tissue surface, internal organ surface) and subsequently freezing that subcutaneous/sub-surface region for localized cryogenic ablation or cryogenic analgesia. In some variations, the percutaneous probe or end effector 106 can comprise a sharp needle, a rounded or blunt needle, a curved shape, or a combination thereof.

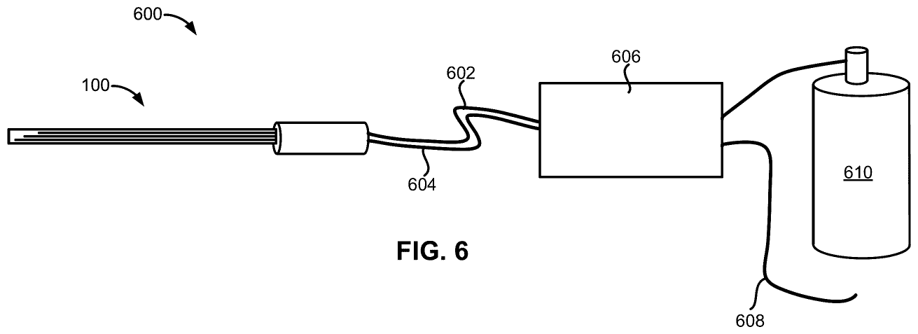

FIG. 6 illustrates a system 600 for using the cryogenic device. The cryogenic device 100 can be coupled to a gas inlet line 602 and a gas outlet line 604 at the handle of the cryogenic device 100. The gas inlet line 602 and the gas outlet line 604 can couple to a generator which controls flow and pressure of the fluid passing through the device 100 as well as serves to monitor the temperature of the device 100 during use. The generator 606 can be coupled to an exhaust line 608 which is coupled to the exhaust space 214 within the elongated shaft 104. The generator can also be coupled to a tank 610 that provides fluid to the device 100. While the end effector 106 of FIG. 4B is shown in this embodiment, it should be understood that any nozzle configuration disclosed herein can be used accordingly.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

It will be understood by one of ordinary skill in the art that the various methods disclosed herein may be embodied in a non-transitory readable medium, machine-readable medium, and/or a machine accessible medium comprising instructions compatible, readable, and/or executable by a processor or server processor of a machine, device, or computing device. The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

We claim:

1. A cryogenic device for ablating tissue, the cryogenic device comprising:
a handle;
an elongated shaft comprising a distal end and a proximal end, wherein the elongated shaft extends from the handle at the proximal end;
an end effector at the distal end of the elongated shaft, wherein the end effector comprises a percutaneous probe;
an inlet line tube within the elongated shaft;
a nozzle at a distal end of the inlet line tube; and
a plate within the inlet line tube, wherein the plate extends radially and comprises a plurality of orifices such that the plurality of orifices faces in a longitudinal direction to deliver fluid through the inlet line tube to the end effector.

2. The cryogenic device of claim 1, wherein the plate is semicircular.

3. The cryogenic device of claim 1, wherein the percutaneous probe comprises a closed needle.

4. The cryogenic device of claim 1, wherein the percutaneous probe comprises a blunt distal end.

5. The cryogenic device of claim 1, further comprising an inner shaft concentrically between the elongated shaft and the inlet line tube.

6. The cryogenic device of claim 5, wherein the inner shaft and the inlet line tube comprising an exhaust path therebetween.

7. The cryogenic device of claim 5, further comprising a vacuum insulated layer between the inner shaft and the elongated shaft.

8. The cryogenic device of claim 5, further comprising an insulative layer between the inner shaft and the elongated shaft, wherein the insulative layer comprises a gas, a liquid, or a solid.

9. The cryogenic device of claim 1, wherein the end effector is configured to cool tissue when cryogenic fluid is introduced to the end effector.

10. A cryogenic device for ablating tissue, the cryogenic device comprising:
a handle;
an elongated shaft comprising a distal end and a proximal end, wherein the elongated shaft extends from the handle at the proximal end;
an inlet line tube within the elongated shaft;
an inner shaft concentrically between the elongated shaft and the inlet line tube;
an end effector at the distal end of the elongated shaft; and
a nozzle at a distal end of the inlet line tube, wherein the nozzle comprises one or more orifices to deliver fluid through the inlet line tube to the end effector, wherein the nozzle comprises a converging portion and a diverging portion.

11. The cryogenic device of claim 10, further comprising a vacuum insulated layer between the inner shaft and the elongated shaft.

12. The cryogenic device of claim 10, wherein the converging portion is proximal to the diverging portion.

13. The cryogenic device of claim 10, wherein the inner shaft and the inlet line tube comprise an exhaust path therebetween.

14. The cryogenic device of claim 10, wherein the end effector is configured to cool tissue when cryogenic fluid is introduced to the end effector.

15. The cryogenic device of claim 10, wherein the one or more orifices are staggered along a length of the end effector.

16. The cryogenic device of claim 10, further comprising an insulative layer between the inner shaft and the elongated shaft, wherein the insulative layer comprises a gas, a liquid, or a solid.

17. A method of ablating tissue; the method comprising:
advancing an elongated shaft percutaneously to a target location, the elongated shaft comprising an inner shaft concentrically within the elongated shaft, the inner shaft comprising an end effector having a needle at a distal end of the inner shaft, wherein a vacuum insulation layer is between the inner shaft and the elongated shaft and is configured to create an insulated portion of the needle such that the insulated portion does not damage tissue in contact with the needle;
positioning the end effector such that a treatment portion of the end effector separate from the insulated portion is configured to ablate tissue at the target location;
introducing a fluid through the elongated shaft and into the end effector; and
ablating the target location with the end effector at the treatment portion.

18. The method of claim 17, further comprising a plate extending radially and comprises a plurality of orifices such that the plurality of orifices faces in a longitudinal direction to deliver fluid to the end effector.

19. The method of claim 18, wherein the plate is semicircular.

20. The method of claim 17, wherein the end effector comprises a closed needle.
